(19) United States
(12) Patent Application Publication
(10) Pub. No.: US 2012/0265209 A1
(43) Pub. Date: Oct. 18, 2012

(54) APPARATUS AND METHODS FOR MIXING MATERIALS

(75) Inventors: Calin Druma, San Jose, CA (US); Bruce Chabansky, Palo Alto, CA (US)

(73) Assignee: Kyphon SARL, Neuchatel (CH)

(21) Appl. No.: 13/086,144

(22) Filed: Apr. 13, 2011

(51) Int. Cl. A61B 17/56

(52) U.S. Cl. 606/93

(57) ABSTRACT

A mixing apparatus comprises a cylindrical body extending between proximal and distal ends. The cylindrical body has a hollow interior and an outlet at the distal end. The mixing apparatus also includes a plunger having an elongated shaft with a distal end, a first mixing plate with a first plurality of holes extending therethrough, and a second mixing plate with a second plurality of holes extending therethrough. The first and second mixing plates have a first relative position, adapted for mixing, in which the first and second plurality of holes are aligned. The mixing plates have a second relative position, adapted for material dispensing, in which the first plurality of holes are blocked by the second mixing plate.

### BACKGROUND

For certain medical procedures, it is desirable or necessary to mix components of a medical preparation together immediately prior to use. It is also desirable to mix components in single use batches, to assure consistency in the delivery of the combined components. Compositions such as bone fillers, bone cements, medical adhesives, dental adhesives, and the like, often rely on mixing syringes combining components and subsequently delivering the combined components to the desired anatomical location.

Kyphoplasty is one example of a procedure that uses mixed compositions to treat vertebral compression fractures and restore vertebral height. Using a minimally invasive approach, one or more compliant balloons is inserted inside a fractured vertebral body. The balloons are inflated such that cancellous bone from the vertebral body is pushed toward the cortical wall, and the resulting cavity is filled with a mixed composition such as polymethylmethacrylate (PMMA). Improved mixing tools and methods are needed for preparing mixed compositions used in kyphoplasty and other medical procedures.

### SUMMARY

Further aspects, forms, embodiments, objects, features, benefits, and advantages of the present invention shall become apparent from the detailed drawings and descriptions provided herein.

In one embodiment, a mixing apparatus comprises a cylindrical body extending between proximal and distal ends. The cylindrical body has a hollow interior and an outlet at the distal end. The mixing apparatus also includes a plunger having an elongated shaft with a distal end, a first mixing plate with a first plurality of holes extending therethrough, and a second mixing plate with a second plurality of holes extending therethrough. The first and second mixing plates have a first relative position, adapted for mixing, in which the first and second plurality of holes are aligned. The mixing plates have a second relative position, adapted for material dispensing, in which the first plurality of holes are blocked by the second mixing plate.

In another embodiment, a method for mixing a medical composition comprises depositing first and second components of the medical composition into a cylindrical body having proximal and distal ends and a hollow interior. The method further includes inserting a plunger into the hollow interior. The plunger includes an elongated shaft with a distal end, a first mixing plate with a first plurality of holes extending therethrough, and a second mixing plate with a second plurality of holes extending therethrough. The method further includes positioning the first plurality of holes in alignment with the second plurality of holes and moving the plunger with an axial reciprocating motion within the cylindrical body to cause portions of the first and second components to move through the aligned first and second plurality of holes.

In another embodiment, a bone cement mixer includes a syringe extending between proximal and distal ends. The syringe has a hollow interior and outlet at the distal end. The mixer also includes a mixing mechanism sized for axial and rotational movement with the syringe. The mixing mechanism including a pair of mixing plates rotatable relative to each other to move between a first position in which a plurality of passages through the mixing plates are aligned to allow flowable bone cement components to pass through the pair of mixing plates and a second position in which the plurality of passages are misaligned to prevent flowable bone cement components from passing through the pair of mixing plates.

### BRIEF DESCRIPTION OF THE DRAWINGS

FIG. 1 illustrates a mixing apparatus according to one embodiment of this disclosure.

FIG. 2a is a top view of a portion of a mixing apparatus in a first configuration according to another embodiment of this disclosure.

FIG. 2b is a cross-sectional view of the portion of the mixing apparatus of FIG. 2a.

FIG. 3a is a top view of the portion of the mixing apparatus of FIG. 2a in a second configuration.

FIG. 3b is a cross-sectional view of the portion of the mixing apparatus of FIG. 3a.

FIGS. 4-6 illustrate the use of a mixing apparatus according to an embodiment of this disclosure.

FIG. 7 is a cross-sectional view of a portion of a mixing apparatus according to another embodiment of this disclosure.

FIG. 8 is a top view of a portion of a mixing apparatus according to another embodiment of this disclosure.

FIG. 9 is a top view of a portion of a mixing apparatus according to another embodiment of this disclosure.

FIG. 10 is a cross sectional view of a portion of a mixing apparatus according to another embodiment of this disclosure.

### DETAILED DESCRIPTION

The present disclosure relates generally to the field of medical composition mixing devices and methods, and more particularly to systems and methods for mixing medical compositions using a convertible mixing and plunging device. For the purposes of promoting an understanding of the principles of the invention, reference will now be made to embodiments or examples illustrated in the drawings, and specific language will be used to describe these examples. It will nevertheless be understood that no limitation of the scope of the invention is thereby intended. Any alteration and further modifications in the described embodiments, and any further applications of the principles of the invention as described herein, are contemplated as would normally occur to one skilled in the art to which the disclosure relates.

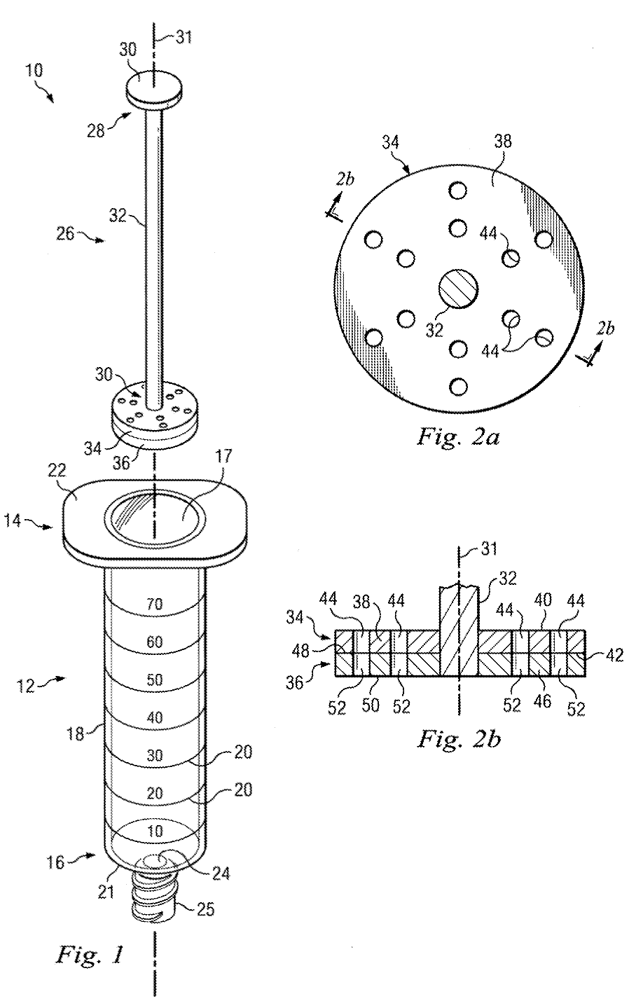

Referring to FIG. 1, a mixing apparatus 10 includes a hollow cylindrical body 12 with a proximal end 14 and a distal end 16. The body 12 includes hollow interior chamber 17, a cylindrical wall 18 with volume markings 20, and a base wall 21 at the distal end 16. The body 12 includes a proximal flange 22 which has an outside diameter greater than that of the wall 18 and which can facilitate handling of the apparatus 10. A distal outlet 24 is formed in the base wall 21. A connector 25 is coupled to the distal outlet 24 to facilitate the coupling of the mixing apparatus 10 to other vessels or instruments. In this embodiment, the body is a syringe and the connector is a Luer connector. It is understood that other types of cylindrical bodies and other types of connectors at the distal outlet may be used. For example, the connector may be an integrally formed nozzle. In this embodiment, the interior chamber may have a volume of approximately 70 ml, but the size of the chosen cylindrical body may depend on the volume of the mixing components and the amount of space needed for mixing action.

The mixing apparatus 10 further include a plunger 26 that functions to both mix components in the interior chamber 17 and to evacuate the mixed material from the interior chamber, as will described in greater detail below. The plunger 26 has a proximal end 28 and a distal end 30. The proximal end 28 includes a handle 30 for controlling the motion of the plunger 26. The plunger 26 includes an elongated shaft 32 extending between the proximal 28 and distal 30 ends along a system axis 31. The shaft 32 may be tubular or solid and may be formed from metals, plastics, ceramics or other materials that provide sufficient rigidity.

A pair of mixing plates 34, 36 are coupled to and extend perpendicularly to the system axis 31 from the distal end of the shaft 32. The mixing plates 34, 36 are circular and have a diameter sized slightly smaller than the inner diameter of the chamber 17 to allow the plates to slide axially within the chamber 17 along the wall 18 of the cylindrical body 12. The plates may be formed from metals, plastics, ceramics, or other materials. Optionally, a sliding seal may extend about the outer perimeter of one or both mixing plates so that the flow of material between the wall of the chamber and the mixing plates is minimized. Alternatively, the mixing plates may have a diameter sufficiently smaller than the inner diameter of the chamber so that material can flow between the edges of the plates and the wall of the chamber.

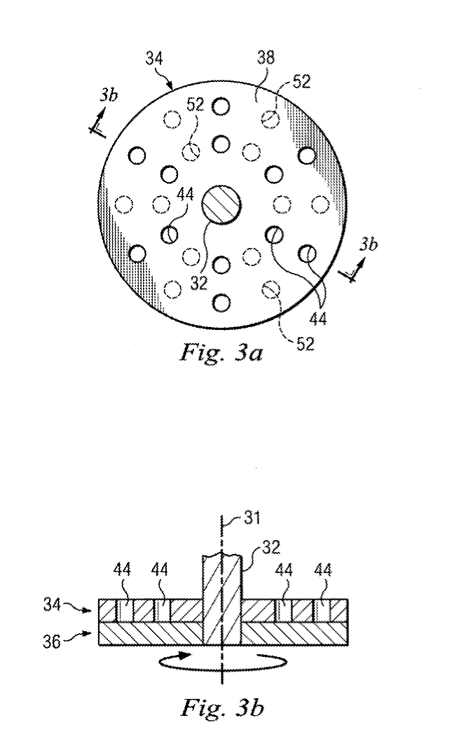

As shown in FIGS. 2a, 2b, 3a, and 3b mixing plate 34 has a plate body 38 with an upper surface 40 and a lower surface 42. Multiple flow holes 44 extend through the plate body 38. Mixing plate 36 has a plate body 46 with an upper surface 48 and a lower surface 50. Multiple flow holes 52 extend through the plate body 46. In this embodiment, the pattern of the flow holes is the same for both plates 34 and 36. The mixing plates 34, 36 are coupled to the shaft 32 such that one or both of the plates are rotatable about the system axis 31.

As shown in FIGS. 2a and 2b, the plates 34, 36 can be arranged in a mixing configuration so that the flow holes 44 and 52 are in alignment to form channels from the upper surface 40 of mixing plate 34 to the lower surface 50 of the plate 36. The channels allows for the passage of material from an area of the chamber 17 below the lower plate 36 to an area of the chamber above the upper plate 34 and vice versa.

The mixing plates 34, 36 can also be arranged in a dispensing configuration. In the dispensing configuration, mixing plate 36 is rotated about the system axis 31 relative to plate 34. As shown in FIGS. 3a and 3b, the rotation of mixing plates relative to each other eliminates the flow channel through the plates, in other words, no channel is formed through the plates between the upper surface 20 and the lower surface 50. Rather, the holes 52 are blocked by the lower surface 42 of the upper plate 34, and the holes 44 are blocked by the upper surface 48 of the lower plate 36. It is understood that the mixing plates may be moved from the mixing configuration to the dispensing configuration by rotation of either mixing plate relative to the other or by the rotation of both plates, with one rotated more than the other.

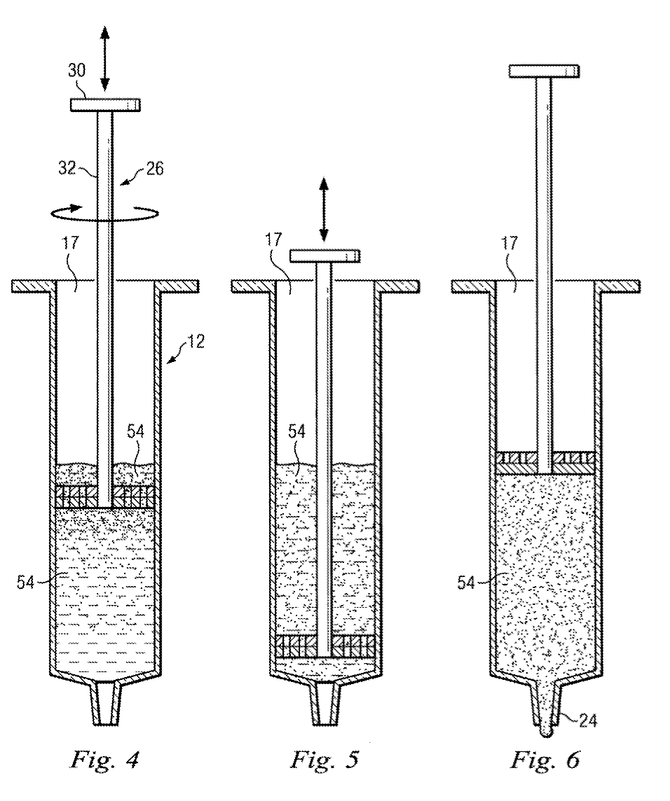

As shown in FIG. 4, the mixing apparatus 10 is assembled by inserting the mixing plates 34, 36 into the cylindrical body 12 with the shaft 32 extending longitudinally within the body along the system axis 31. The shaft is sized such that the handle 30 protrudes from the proximal end of the body 12 when the distal end 30 of the plunger 26 extends into the distal end 16 of the cylindrical body. The plunger 26 can rotate and can move in an axial reciprocating motion within the cylindrical body 12.

The mixing apparatus 10 may be used for mixing and dispensing medical compositions such as bone fillers, bone cements, medical adhesives, dental adhesives, and the like. The medical compositions may be formed of multiple components, including liquids, powders, solids, pasty materials, and viscous materials. Commonly, the mixing apparatus may be used to mix a two-part composition comprising a liquid and a power component. Powder components may include autograft tissue, allograft tissue, demineralized bone matrix, calcium phosphate compositions, or polymer cement components. Liquid components may include a buffered aqueous solution, glycerol, or a monomer cement component. The mixing apparatus 10 may be particularly suitable for use with PMMA compositions such as KYPHON HV-R® Bone Cement or compositions that contain PMMA and hydroxyapatite (HA) such as KYPHON ActivOs™10 bone cement offered by Medtronic, Inc. of Minneapolis, Minn. It is understood that the examples provided are not limiting and the mixing apparatus of this disclosure may be used to combine components for any medical compositions.

Methods of using the mixing apparatus 10 will now be described. The mixing apparatus is initially arranged in a mixing configuration. To prevent the flow of material from the distal outlet 24, the outlet is blocked by a cap, a mixing stand, a frangible membrane or other occluding devices known in the art. Two or more components of a chosen medical composition 54 are deposited in the interior chamber 17 of the cylindrical body 12. The components may be added separately or may be premixed and introduced to the chamber 17 together in a nonhomogenous mixture. The mixing plates are arranged as shown in FIGS. 2a and 2b such that the holes 44 align with the holes 52 to form multiple channels through the plates 34, 36. As shown in FIGS. 4 and 5, the plunger 26 is inserted into the interior chamber 17 and the components of the medical composition are mixed by a reciprocating movement of the mixing plates 34, 36 axially along the system axis 31. The plunger 26 can also be rotated within the chamber 17. The movement of the plunger 26 within the chamber 17 causes the components of the composition 54 to flow through the holes 44 and 52. When the mixing plates are moved from the proximal end of the chamber toward the distal end of the chamber, the components of the composition flow through the holes toward the proximal end of the chamber. When the mixing plates are moved from the distal end of the chamber toward the proximal end, the components of the composition flow through the holes toward the distal end of the chamber. Repeated translational and/or rotational motion of the plunger 26 within the chamber 17 is used to mix the components and create a generally homogenous medical composition 54.

Referring now to FIG. 6, the plunger may be reconfigured from the mixing configuration to a dispensing configuration. The distal outlet 24 is opened and the connector 25 is coupled to a material delivery device or other vessel so that the mixed medical compound 54 can be transferred from the chamber 17. The plunger 26 is withdrawn axially from the homogenous medical composition 54 until substantially all of the medical composition is located between the lower plate 36 and the nozzle outlet 24. The plunger 26 is changed from a mixing instrument to a dispensing instrument by changing the configuration of the mixing plates 34, 36 from an arrangement in which flow holes 44 are aligned with flow holes 52 (FIG. 2b) to an arrangement in which flow holes 44 are misaligned from the holes 52 and blocked by the plate body 46 (FIG. 3b). With the flow holes 44 and 52 blocked such that the mixed medical composition does not flow through the pair of plates 34, 36, axial force is applied to the handle 30 to cause the mixing plates 34, 36 to advance through the chamber 17 to push the medical composition from the chamber through the distal outlet 24. The amount of material dispensed may be gauged by reference to the markings 20.

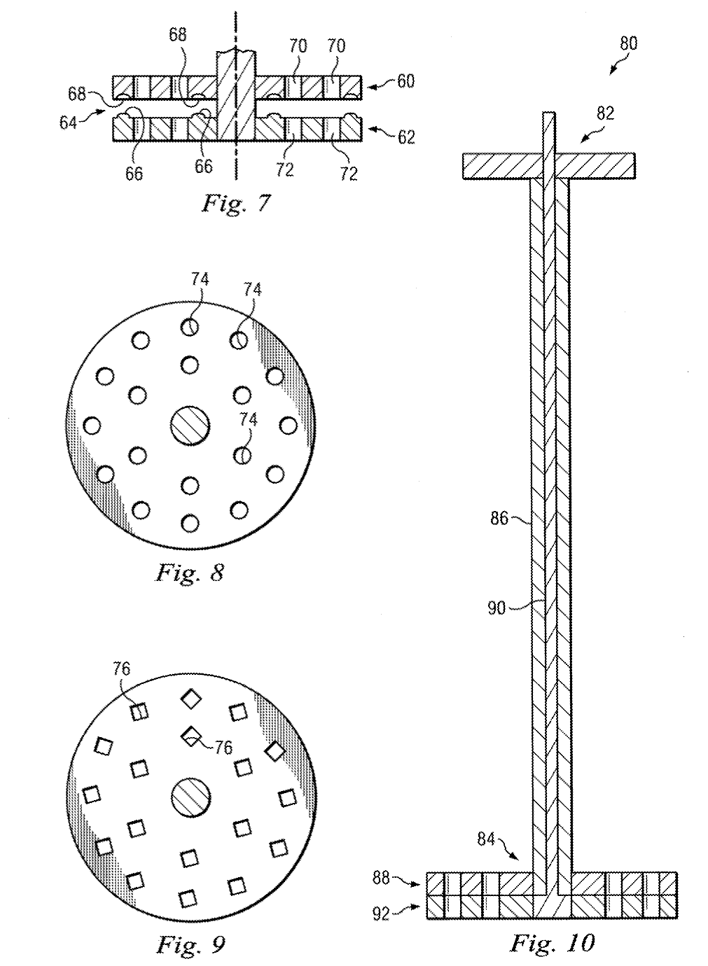

Referring now to FIG. 7, in some embodiments a releasable locking mechanism may be used to hold the mixing plates is fixed relationship to each other in the mixing and in the dispensing configurations. For example, an upper mixing plate 60 may be held in a fixed relationship to a lower mixing plate 62 using a locking mechanism 64 that includes one or more projections 66 from the lower mixing plate 62 sized for receipt within one or more depressions 68 in the upper mixing plate 60. In a mixing configuration, in which flow holes 70 and 72 are aligned to form through channels, the projections 66 are seated in the depressions 68 to prevent the mixing plate 60 from rotating with respect to the mixing plate 62. To move the plates from a mixing configuration to a dispensing configuration, the projections 66 are removed from the depressions 68, and the plates 60 and 62 are rotated relative to one another until the flow holes 70, 72 are blocked by the opposite plate. In this dispensing configuration, the projections 66 are seated into a different set of depressions (not shown) to lock the plates 60, 62 in the dispensing position. Optionally, the locking mechanism may include features, in addition to or other than the projection/depression locking mechanism, such as spring-loaded mechanisms, threaded mechanisms, ratchet mechanisms, or other mechanisms known in the art to apply force to hold the mixing plates together.

In the embodiment of FIGS. 2a-3b, the mixing plates are arranged such that the upper surface 48 of the lower mixing plate 36 abuts the lower surface 42 of the upper mixing plate 34. In alternative embodiments, the mixing plates may be spaced apart. With spaced apart plates, in the mixing configuration, the flow holes may be aligned or misaligned since the material composition may flow through flow holes and through the space between the plates to move from a distal end to a proximal end of the chamber 17.

In alternative embodiments, the mixing plates may have a flow hole pattern 74, as shown in FIG. 8, with many flow holes or a flow hole pattern 76, as shown in FIG. 9, with flow holes on a different shape. It is understood that the flow hole patterns may include more or fewer holes and holes of different shapes and sizes. In some embodiments, the flow hole patterns on the upper and lower mixing plates may be different. The flow hole patterns may also permit more than one mixing configuration, such one configuration in which all of the flow holes are aligned to be open and another configuration in which a portion of the flow holes are blocked and a portion of the flow holes are open for mixing.

As shown in FIG. 10, the shaft of the plunger may include multiple components that allow the configuration of the mixing plates to be changed by a user through the manipulation of components at the proximal end of the plunger. For example, the plunger 80 has a proximal end 82 and a distal end 84. The plunger 80 includes an elongated outer shaft 86 coupled to an upper mixing plate 88 and an elongated inner shaft 90 coupled to a lower mixing plate 92. To move the upper mixing plate 88 with respect to the lower mixing plate 92, the inner shaft 90 is rotated within the outer shaft 86. Other systems known in the art may, alternatively, be used to remotely adjust the rotation of the mixing plates relative to one another.

While the present invention has been illustrated by the above description of embodiments, and while the embodiments have been described in some detail, it is not the intention of the applicant to restrict or in any way limit the scope of the invention to such detail. Additional advantages and modifications will readily appear to those skilled in the art. Therefore, the invention in its broader aspects is not limited to the specific details, representative apparatus and methods, and illustrative examples shown and described. Accordingly, departures may be made from such details without departing from the spirit or scope of the applicant's general or inventive concept. In the claims, means-plus-function clauses are intended to cover the structures described herein as performing the recited function and not only structural equivalents, but also equivalent structures.

What is claimed is:

1. A mixing apparatus comprising:
a cylindrical body extending between proximal and distal ends with a hollow interior and an outlet at the distal end and
a plunger including an elongated shaft with a distal end, a first mixing plate with a first plurality of holes extending therethrough, and a second mixing plate with a second plurality of holes extending therethrough,
wherein the first and second mixing plates have a first relative position, adapted for mixing, in which the first and second plurality of holes are aligned and a second relative position, adapted for material dispensing, in which the first plurality of holes are blocked by the second mixing plate.

2. The mixing apparatus of claim 1 wherein the first mixing plate is rotatable with respect to the second mixing plate to transition from the first relative position to the second relative position.

3. The mixing apparatus of claim 1 wherein the plunger further includes a locking mechanism for preventing rotation of the first mixing plate with respect to the second mixing plate.

4. The mixing apparatus of claim 1 wherein in the first relative position, the first mixing plate is in contact with the second mixing plate.

5. The mixing apparatus of claim 1 further comprising a seal between at least one of the first and second mixing plates and the cylindrical body.

6. The mixing apparatus of claim 1 further comprising an device for occluding the outlet when the first and second mixing plate are in the first relative position.

7. The mixing apparatus of claim 1 wherein the plunger is axially translatable within the cylindrical body.

8. The mixing apparatus of claim 1 wherein the plunger is rotatable within the cylindrical body.

9. The mixing apparatus of claim 1 wherein the first and second mixing plates extend generally perpendicular to the elongated shaft.

10. The mixing apparatus of claim 1 wherein the outlet is coupled to a luer connector.

11. A method for mixing a medical composition comprising:
depositing first and second components of the medical composition into a cylindrical body having proximal and distal ends and a hollow interior;
inserting a plunger into the hollow interior, wherein the plunger includes an elongated shaft with a distal end, a first mixing plate with a first plurality of holes extending therethrough, and a second mixing plate with a second plurality of holes extending therethrough;
positioning the first plurality of holes in alignment with the second plurality of holes; and
moving the plunger with an axial reciprocating motion within the cylindrical body to cause portions of the first and second components to move through the aligned first and second plurality of holes.

12. The method of claim 11 wherein the step of positioning includes locking the first mixing plate with respect to the second mixing plate to prevent relative rotational movement.

13. The method of claim 11 further comprising repositioning the second mixing plate to block the first plurality of holes.

14. The method of claim 13 further comprising after repositioning the second mixing plate, moving the plunger axially within the cylindrical body to dispense the medical composition from the distal end of the cylindrical body.

15. The method of claim 11 further comprising moving the plunger with a rotational motion within the cylindrical body.

16. The method of claim 10 further comprising dispensing the medical composition into a bone filler device.

17. A bone cement mixer comprising:
a syringe extending between proximal and distal ends, the syringe having a hollow interior and outlet at the distal end and
a mixing mechanism sized for axial and rotational movement with the syringe, the mixing mechanism including a pair of mixing plates rotatable relative to each other to move between a first position in which a plurality of passages through the mixing plates are aligned to allow flowable bone cement components to pass through the pair of mixing plates and a second position in which the plurality of passages are misaligned to prevent flowable bone cement components from passing through the pair of mixing plates.

18. The bone cement mixer of claim 17 further comprising a locking mechanism to prevent relative rotation between the mixing plates.

19. The bone cement mixer of claim 17 wherein the mixing mechanism further includes a shaft to which the pair of mixing plates is coupled.

20. The bone cement mixer of claim 19 wherein the shaft includes a proximal handle.
